(19) United States
(12) Patent Application Publication
(10) Pub. No.: US 2017/0003281 A1
(43) Pub. Date: Jan. 5, 2017

(54) RESPONSIVE HYDROGEL FOR THE DETECTION OF BIOMOLECULES

(71) Applicant: FRAUNHOFER-GESELLSCHAFT ZURFORDERUNG DER ANGEWANDTEN FORSCHUNG E.V., Munchen (DE)

(72) Inventors: Andre Laschewsky, Potsdam (DE); Erik Wischerhoff, Potsdam (DE); Martin Sutterlin, Munchen (DE); Jean-Philippe Couturier, Eschborn (DE)

(21) Appl. No.: 15/109,248

(22) PCT Filed: Dec. 12, 2014
(86) PCT No.: PCT/EP2014/077554

§ 371 Date: Jun. 30, 2016

(30) Foreign Application Priority Data
Jan. 8, 2014 (DE) 10 2014 200 135.8

(51) Int. Cl. G01N 33/544; C08J 3/075; B01J 13/00

(52) U.S. Cl. CPC G01N 33/544; B01J 13/0065; C08J 3/075; C08J 2300/14; C08J 2333/06

(57) ABSTRACT

The present invention relates to a responsive hydrogel which is chemically crosslinked, has a porous photonic crystal structure and contains biomolecule-specific detection groups.

The present invention relates to a hydrogel that can be used for the detection of biomolecules.

Biosensors and rapid tests play an important role in medicine for diagnosis of diseases and for determining a physiological state. The basis is the identification and detection of biomarkers. According to the prior art, the detection step is usually carried out by using a labeling agent (e.g., a radio-labeled secondary antibody) or the species to be detected itself must be labeled (e.g., fluorescent labeling of DNA fragments). There are also a number of biosensors, e.g. based on surface plasmon resonance or interference phenomena, which do not mark the detected species. However, these require elaborate instrumentation. Desirably the direct optical detection of biological species should be performed without the use of labeling reagents and without any additional instrumentation.

WO 03/025538 A2 describes a sensor for determining the concentration of a chemical species, wherein the sensor includes a periodic array of colloidal particles in a hydrogel matrix. S. A. Asher et al., Anal. Chem., 70 (1998), 780-791, describe a hydrogel film, wherein periodically arranged colloidal particles are present. Since larger biomolecules cannot diffuse through these structures, an analytical detection of these structures by such sensor systems is not possible. However, in particular the detection of such large molecules or molecular aggregates, such, for example, proteins, DNA, or viruses would be particularly desirable because of their high biological and medical relevance.

An object of the present invention is to provide a sensory material, which allows the detection of biomolecules and biological substances by a simple detection method, in particular without labeling reagents, secondary antibody and expensive instrumentation.

The object is achieved by a responsive hydrogel which is chemically crosslinked, has a porous photonic crystal structure and contains biomolecule-specific detection groups.

As will be described in the following in more detail, hydrogels are accessible in the form of a porous photonic crystal by a template synthesis, wherein at first a photonic crystal of colloidal particles is provided as a template, and a hydrogel is polymerized in the interstices between these colloidal particles, followed by the removal of the colloidal particles in order to obtain a porous photonic crystal structure. Surprisingly it has been shown in the context of the present invention, that the porosity of such a photonic crystal is sufficient to enable the diffusion of even larger molecules, in particular biomolecules, such as e.g. biooligomers, biopolymers or biological particles. Thus, since it was found that with the inventive hydrogel structure the detection of biomolecules, in particular larger biomolecules such as biooligomers, biopolymers or biological particles is possible, the hydrogel contains biomolecule-specific detection groups. Furthermore, it has been shown in the present invention that a porous photonic crystal, which is formed by a responsive hydrogel (i.e., a hydrogel, which can change its swelling behavior under the influence of an external stimulus), may exhibit a color change even in the wavelength range of visible light and can therefore make an elaborate instrumentation superfluous.

A hydrogel is generally defined as a three-dimensional network, which is not soluble in water, but imbibes water and thus swells. In principle, the crosslinking of the polymer chains can be carried out physically or chemically in hydrogels. In a physically crosslinked gel, the network nodes are formed by entanglement through loops or hooks of long polymer chains among each another. Network nodes of physical interactions such as electrostatic interactions may be formed as well. In chemically crosslinked gels, the nodal points are formed by covalent bonds between the polymer chains.

In the present invention, the hydrogel is chemically crosslinked. This ensures that the porous photonic crystal structure of the hydrogel has a sufficient stability.

The hydrogel according to the invention is a responsive hydrogel. According to the skilled person responsive hydrogels are such hydrogels which swell under the influence of an external stimulus (i.e. under the influence of an external, changing parameter) by imbibing fluid or alternatively may collapse when the liquid escapes. These transitions occur preferably in a reversible manner.

Such hydrogels are basically known to the person skilled in the art and are referred to as “stimuli-responsive hydrogels” or “switchable hydrogels”.

For a responsive hydrogel for example, those materials are suitable which have a volume phase transition. The volume phase transition may be based on a lower and/or an upper critical solution temperature, however, there is no dissolution of the polymer in the solvent in the case of crosslinked systems. In such a system, for example, the temperature may act as a switch. In this context, this is referred to as thermoresponsive hydrogel. The release of liquid takes place when exceeding or undercutting a threshold temperature. Such systems can be modified by the incorporation of additional groups which modify their hydrophilicity upon application of another stimulus than the temperature. By responding to that other stimulus various other switching phenomena can be realized in a certain temperature interval. Such systems are, for example, described by M. Irie, Adv. Polym. Sci. 110, 43-65 (1993). Thus, e.g. pH, ionic strength, light or chemical reactions are used as switches. With a suitable design of the system even complex biological macromolecules such as proteins can act as a stimulus. Such systems are, for example, described by J. Buller et al., Polym. Chem. 2, 1486-1489 (2011).

Examples of groups that have the ability to elicit switching by the influence of light are azobenzenes (R. Kroger et al., Macromol. Chem. Phys. (1994) 195, 2291-2298) or spiropyrans (Edahiro et al., Biomacromolecules (2005) 6, 970-974). Examples of groups having the ability to switch via the pH are amino or carboxyl groups. An example of the ability to switch via a chemical reaction is described in P. Mi et al., Macromol. Rapid Commun (2008) 29, 27-32.

In preferred embodiments, the responsive hydrogels are those which swell or shrink by an external stimulus (i.e., by changing an external parameter), which stimulus is selected from temperature, pH, ionic strength, ionic species, electromagnetic radiation (especially light), a chemical reaction, the presence (addition or replacement) of chemical (in particular low molecular weight) or biochemical reagents or an action by biomolecules such as proteins, or combinations thereof. Examples of polymers that can be used as a basic structure for hydrogels having an upper critical solution temperature can be found in J. Seuring et al., Macromolecules (2012), 45, 3910-3918.

As described in more detail below the responsive hydrogel of the present invention contains biomolecule-specific detection groups. It is preferred that the hydrogel either swells by connecting the biomolecules to the specific detection groups or shrinks. In a preferred embodiment, the responsive hydrogel exhibits a volume phase transition by binding of the biomolecules to the specific detection groups. With such a volume phase transition the responsive hydrogel exhibits under isothermal conditions a sudden change in volume as a result of the connection of biomolecules and a consequent change in the hydrophilic or hydrophobic character.

By the volume change of the responsive hydrogel, which is caused by the connection of the biomolecule to the specific detection groups, the size of the unit cell of the photonic crystal and thus also the peak maximum of the Bragg reflection is changed. This peak shift can be used for the analytical detection of the biomolecules.

Suitable monomer units for responsive hydrogels are known to the person skilled in the art. For example, the responsive hydrogel contains one or more of the following monomer units (and, optionally, other monomer units for more precise fine tuning of the material properties):

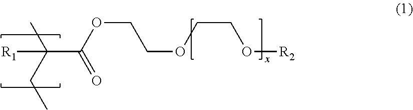

wherein

R₁═H, alkyl such as e.g. C₁₋₄-alkyl, preferably —CH₃;

R₂═H, alkyl such as e.g. C₁₋₄-alkyl, —(CH₂)ₙ—COOH with n=1 to 12 or a salt thereof;

x=0-50, more preferred 1-50 or 1-20,

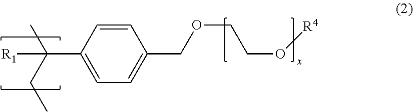

wherein

R₁═H, alkyl such as e.g. C₁₋₄-alkyl, preferably —H;

R₄═H, alkyl such as e.g. C₁₋₄-alkyl, —(CH₂)ₙ—COOH with n=1 to 12 or a salt thereof;

x=0-50, more preferred 1-50 or 3-20,

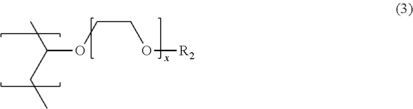

wherein

R₂═H, alkyl such as e.g. C₁₋₄-alkyl, —(CH₂)ₙ—COOH with n=1 to 12 or a salt thereof;

x=0-50, more preferred 1-50 or 2-20,

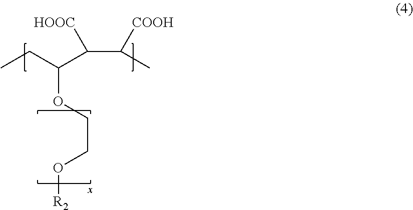

wherein

R₂═H, alkyl such as e.g. C₁₋₄-alkyl, —(CH₂)ₙ—COOH with n=1 to 12 or a salt thereof;

x=0-50, more preferred 1-50 or 2-20,

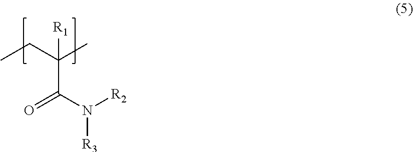

wherein

R₁═H, alkyl such as e.g. C₁₋₄-alkyl;

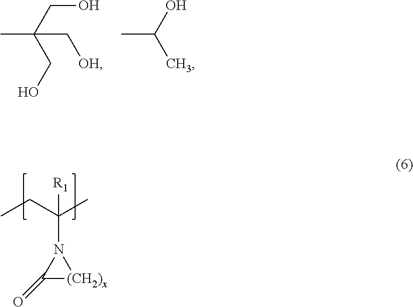

R₂, R₃ independently of each other=H, alkyl such as e.g. C₁₋₄-alkyl, allyl,

wherein

R₁═H, alkyl such as e.g. C₁₋₄-alkyl, preferably H;

x=1-6, more preferred 3-4,

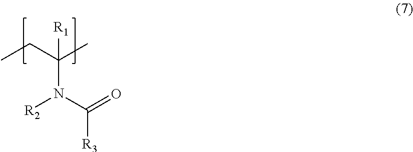

wherein

R₁, R₂, R₃ independently of each other=H, alkyl such as e.g. C₁₋₄-alkyl;

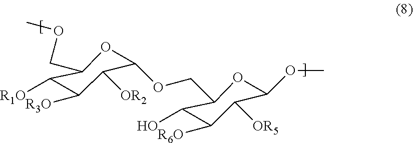

R₁═H or a branch of the polysaccharide chain;

R₂, R₃, R₅, R₆ independently of each other=H, alkyl such as e.g. C₁₋₄-alkyl, allyl, —(CH₂)ₙ—COOH or a salt thereof,

(9) polysaccharide units, such as e.g. carboxymethylcellulose units, hydroxyethyl starch units,

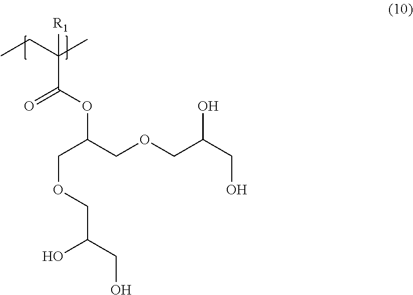

wherein

R₁═H, alkyl such as e.g. C₁₋₄-alkyl, preferably —CH₃,

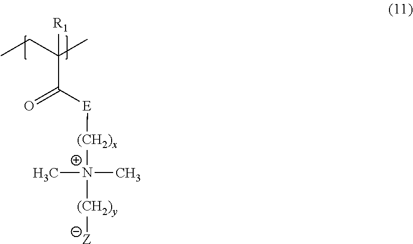

wherein

R₁═H, alkyl such as e.g. C₁₋₄-alkyl, preferably —CH₃,

E=O or NH,

x, y independently of each other may acquire values of from 1 to 12, Z═SO₃, COO, or PO₃.

The respective monomer units may be randomly distributed along the polymer chain or may occur in block form. It is preferably a random copolymer.

In a preferred embodiment, the responsive hydrogel contains the following monomer units (1a) and (1b) (and optionally additional monomeric units for accurate fine-tuning of the material properties):

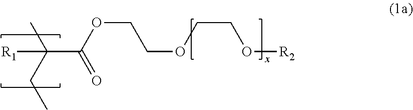

wherein

R₁═H, alkyl such as e.g. C₁₋₄-alkyl, preferably —CH₃;

R₂═H, alkyl such as e.g. C₁₋₄-alkyl, —(CH₂)ₙ—COOH with n=1 to 12 or a salt thereof,

x=0 or 1 or 2,

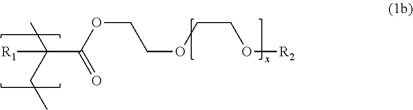

wherein

R₁═H, alkyl such as e.g. C₁₋₄-alkyl, preferably —CH₃,

R₂═H, alkyl such as e.g. C₁₋₄-alkyl, —(CH₂)ₙ—COOH with n=1 to 12 or a salt thereof,

x=3-50, more preferred 3-20 or 4-10.

When using the monomer units (1a) and (1b) it has been proven to be advantageous that the responsive hydrogel is not completely collapsed in the range of room temperature, i.e. a temperature being preferred for the analytical detection of biomolecules (i.e. the degree of swelling is not 0) and therefore the specific detection groups have good accessibility for the biomolecules to be detected. When the biomolecules to be detected bind to the detection groups, there is a significant change in volume and thus also to a very significant shift of the peak maximum of the Bragg reflection.

The proportion of monomer units (1a) and (1b) can vary over a wide range. For example, the monomer units (1a), based on the total amount of the monomers, may be present in an amount of 30-90 mol %, more preferred 50-80. The monomer units (1b) can be present, for example, in an amount of 2-40 mol %, preferably in an amount of 5-35 mol %, based on the total amount of monomers.

In order to allow covalent attachment of the biomolecule-specific detection group to the hydrogel, it may be preferred that some or all of the monomer units (1a) in the side chain have a reactive group, in particular OH, or COOH. In one of these preferred embodiments, the monomer units (1a) have the following structure:

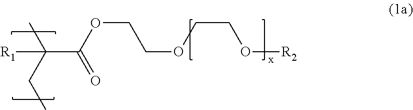

wherein

R₁═H, alkyl such as e.g. C₁₋₄-alkyl, preferably —CH₃

R₂═H, —(CH₂)ₙ—COOH with n=1 to 12 or a salt thereof;

x=0 or 1 or 2.

Alternatively, the monomer units (1a) are a mixture of at least two different monomer units (i) and (ii), which have the following structures:

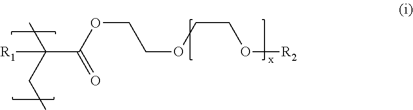

wherein

R₁═H, alkyl such as e.g. C₁₋₄-alkyl, preferably —CH₃,

R₂═H, —(CH₂)ₙ—COOH with n=1 to 12 or a salt thereof

x=0 or 1 or 2,

and

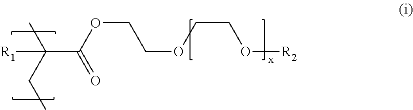

wherein

R₁═H, alkyl such as e.g. C₁₋₄-alkyl, preferably —CH₃,

R₂=alkyl such as e.g. C₁₋₄-alkyl,

x=0 or 1 or 2.

As already mentioned above, the responsive hydrogel of the present invention is chemically crosslinked.

The skilled person generally knows, as to how hydrogels can chemically be crosslinked.

It is preferred in the present invention that chemical crosslinking is effected by the preparation of the hydrogel in the presence of crosslinkable monomers and the crosslinkable monomers are preferably present in an amount of 2-20 mol %, based on the total amount of monomers. It has been proven to be advantageous with this amount of crosslinking monomers that the hydrogel and thus the porous photonic crystal structure have a sufficient stability, but on the other hand, that the specific detection groups for the biomolecules to be detected are still easily accessible.

The crosslinkable monomers may be photocrosslinkable or thermally crosslinkable monomers. For this purpose, suitable monomers are basically known to those skilled in the art.

For example the crosslinkable monomers may be multifunctional (e.g., bi-, tri- or tetra-functional) monomers, i.e. monomers having two or more functional groups.

Suitable multifunctional monomers are e.g. multifunctional acrylic, methacrylic, vinyl or allyl monomers.

For example, di(meth)acrylates or tri(meth)acrylates, which optionally can also be ethoxylated, may be used as multi-functional crosslinker monomers.

If ethoxylated di (meth) acrylates are used as crosslinking monomers, they may have the following chemical formula:

H₂C═C(R₁)—C—O—[CH₂—CH₂—O]ₙ—C(O)—C(R₂)═CH₂

wherein

R₁ and R₂ independently of each other are H or methyl and

n=1-5000, preferably 1-100 or 1-30.

Preferably R₁ and R₂ are the same, i.e. R₁═R₂═H or methyl.

Suitable photocrosslinkable monomers (i.e. monomers which cause the crosslinking of adjacent polymer chains by means of a photochemical reaction) are known to those skilled in the art. Such photocrosslinkable monomers contain a photoreactive group, for example, a benzophenone group, an acetophenone group, a diazirine group or an azide group.

The responsive hydrogel of the present invention has a porous photonic crystal structure.

In the present invention, the term “photonic crystal” is used in its usual meaning as known to the skilled person and therefore the term refers to a material having a spatially periodically varying refractive index, wherein the period length is comparable to the wavelength of light and the material exhibits a Bragg reflection at a defined wavelength. The material itself, forming the photonic crystal does not have to be a crystalline one. It is crucial that the material (in the case of the present invention, the responsive hydrogel) is spatially arranged such that a periodically varying refractive index results. If this period length changes, for example, because the photonic crystal shrinks or swells, then the peak position of the Bragg reflection changes too. If the displacement of the peak positions of the Bragg reflection occurs in the visible wavelength range of light and if the extent of shift is sufficiently large, then the change in volume of the photonic crystals can be perceived with the naked eye. Opals are a prominent example of a photonic crystal or a material having a photonic crystal structure.

Because of the porosity of the photonic crystal structure formed by the hydrogel a diffusion even of larger biomolecules is easily possible in this structure and thus all of the specific detection groups of the hydrogel are basically accessible.

As will be described in greater detail below, such a porous photonic crystal structure is obtained firstly by forming a photonic crystal from colloidal particles, which are preferably monodisperse, and by forming in the interstices of this colloidal particles a chemically crosslinked hydrogel. Preferably, the colloidal particles are packed so densely, that a particle touches its neighboring particles as much as possible. Thus preferably the packing of the colloidal particles is as close as possible. At the contact surfaces of adjacent particles no polymer is formed. Then, the colloidal particles are removed, for example, by a suitable solvent, and a porous photonic crystal structure remains. The porous photonic crystal structure of the hydrogel according to the invention is thus obtained by a template induced manufacturing method, wherein a photonic crystal of colloidal particles acts as the template. The porous photonic crystal structure of the hydrogel is actually the negative of the structure of the photonic template crystals.

It is preferable that the porous photonic crystal structure has an inverse opal structure.

As a consequence of the manufacturing process using photonic template crystals of colloidal particles, the porous photonic crystal structure exhibits cavities or voids after the removal of these colloidal particles. There are interconnected voids, so that a diffusion of the biomolecules to be detected into the porous photonic crystal structure is possible and the specific detection groups of the hydrogel are easily accessible.

The diameter of the cavities or voids of the porous photonic crystal structure can be controlled by the size of the colloidal particles of photonic template crystals. It is preferable that these particles are monodisperse particles. Preferably, the colloidal particles of the photonic template crystals have a coefficient of variation of <20%, more preferably <10% or even <5%.

Suitable colloidal particles for the manufacture of a photonic crystal are commercially available or can be obtained via standard manufacturing processes, which are well-known to those skilled in the art.

The average diameter of colloidal, preferably monodisperse particles of the photonic template crystals and thus also of the cavities of the porous photonic crystal structure may be varied over a wide range. For example the average diameter may be in the range of 600 to 100 nm, preferably from 500 to 150 nm (for example, determined by scanning electron microscopy). With a suitable choice of the average diameter, a Bragg peak of the porous photonic crystal can be obtained in the visible wavelength range. This in turn allows an analytical detection of the biomolecules in the wavelength range of visible light.

In the context of the present invention it has also been found that the cavities or voids produced via such a template synthesis are connected together (i.e. interconnected) and the passages between these cavities or voids are sufficiently large to allow for the detection of large biomolecules.

Therefore, in a preferred embodiment, the biomolecule-specific detection groups are those which are suitable for the detection of biooligomers, biopolymers or biological particles. Exemplary biomolecule-specific detection groups which may be mentioned in this regard are antibodies, F_{ab}-fragments of antibodies, enzymes, enzyme fragments, coenzymes, peptides, prosthetic groups, aptamers, single strands of DNA and RNA.

Exemplary biooligomers are oligopeptides, oligosaccharides, oligonucleotides.

Exemplary biopolymers are polypeptides, proteins, polysaccharides, polynucleotides, nucleic acids.

Exemplary biological particles are viruses.

The biomolecule-specific detection groups are preferably attached to the hydrogel via a covalent linkage.

The covalent binding of the biomolecule-specific detection groups may be realized in the hydrogel by carrying out polymerization in the presence of monomer compounds which already contain such a biomolecule-specific detection group. Alternatively, it is also possible that in this polymerization firstly a monomer compound having an organic functional group (such as —OH or —COOH) is present, and only after the polymerization, these organic functional groups are reacted with a compound containing the biomolecule-specific detection group.

According to a further aspect, the present invention relates to a process for preparing a chemically crosslinked, responsive hydrogel having a porous photonic crystal structure, comprising:
providing a photonic template crystal of colloidal particles,
introduction of monomers into the interstices being present between the colloidal particles,
polymerization of the monomers, thereby obtaining a chemically crosslinked, responsive hydrogel,
removal of the colloidal particles of the photonic template crystal, thereby obtaining while maintaining the porous photonic crystal structure.

Suitable colloidal particles for the formation of photonic crystals are basically known to those skilled in the art. Preferred are monodisperse particles. For example the colloidal particles can have a coefficient of variation of <20% or <10% or even <5%. Both inorganic particles (for example, SiO₂ particles) and organic polymer particles may be used. These particles have to be selected in a manner that they can be removed again, for example, under the action of a solvent and/or thermal action.

Such colloidal, preferably monodisperse particles are available by means of conventional manufacturing methods known in the art or are commercially available.

The photonic template crystals are provided via conventional manufacturing methods known in the art. Preferably, a dispersion of colloidal particles is applied to a substrate and allowed to slowly evaporate the liquid dispersion medium. The colloidal particles are deposited on the substrate in a periodically uniform arrangement, thereby forming the photonic template crystal.

Regarding suitable monomers for the formation of a chemically crosslinked, responsive hydrogel it may be referred to the above statements.

In a preferred embodiment, the monomers comprise the following compounds (a1) and (a2) (and optionally other compounds for fine tuning the desired end properties):

H₂C═C(R₁)—C(O)—O—CH₂—CH₂—[CH₂—CH₂—O]ₓ—R₂ (a1)

wherein

R₁═H, alkyl such as e.g. C₁₋₄-alkyl, preferably —CH₃,

R₂═H, alkyl such as e.g. C₁₋₄-alkyl, —(CH₂)ₙ—COOH with n=1 to 12 or a salt thereof,

x=0 or 1 or 2,

H₂C═C(R₁)—C(O)—O—CH₂—CH₂—[CH₂—CH₂—O]ₓ—R₂ (a2)

wherein

R₁═H, alkyl such as e.g. C₁₋₄-alkyl, preferably —CH₃,

R₂═H, alkyl such as e.g. C₁₋₄-alkyl, —(CH₂)ₙ—COOH with n=1 to 12 or a salt thereof, more preferred C₁₋₄-alkyl,

x=3-50, more preferred 3-20 or 4-10.

The proportion of the monomers (a1) and (a2) can vary over a wide range. For example, the monomers (a1) may be present in an amount of 30-90 mol %, more preferably 50-80 mol %, based on the total amount of the monomers. For example the monomer units (a2) may be present in an amount of 2-40 mol %, more preferably 5-35 mol % based on the total amount of the monomers.

In order to allow covalent attachment of a biomolecule-specific detection group to the hydrogel, it may be preferred that some or all of the monomers (a1) in the side chain have a reactive group, in particular OH, or COOH. In one of these preferred embodiments, the monomers (a1) have the following structure:

H₂C═C(R₁)—C(O)—O—CH₂—CH₂—[CH₂—CH₂—O]ₓ—R₂

wherein

R₁═H, alkyl such as e.g. C₁₋₄-alkyl, preferably —CH₃,

R₂═H, —(CH₂)ₙ—COOH with n=1 to 12 or a salt thereof,

x=0 or 1 or 2.

Alternatively, the monomers (a1) may be a mixture of at least two different monomers (a1.1) and (a1.2), which have the following structures:

H₂C═C(R₁)—C(O)—O—CH₂—CH₂—[CH₂—CH₂—O]ₓ—R₂ (a1.1)

wherein

R₁═H, alkyl such as e.g. C₁₋₄-alkyl, preferably —CH₃,

R₂═H, —(CH₂)ₙ—COOH with n=1 to 12 or a salt thereof,

x=0 or 1 or 2;

and

H₂C═C(R₁)—C(O)—O—CH₂—CH₂—[CH₂—CH₂—O]ₓ—R₂ (a1.2)

wherein

R₁═H, alkyl such as e.g. C₁₋₄-alkyl, preferably —CH₃,

R₂=alkyl such as e.g. C₁₋₄-alkyl,

x=0 or 1 or 2.

As described above, in the present invention, the chemical crosslinking is effected by the preparation of the hydrogel in the presence of crosslinkable monomers and the crosslinkable monomers are preferably present in an amount of 2-20 mol %, based on the total amount of monomers.

With regard to preferred crosslinkable monomers reference is made to the above statements.

Suitable polymerization conditions for the conversion of the monomers to the hydrogel are known to the person skilled in the art.

As already described above, the covalent bond of the biomolecule-specific detection groups can be realized in the hydrogel if monomer compounds are present during the polymerization which already contain such a biomolecule-specific detection group. Alternatively, it is also possible in this polymerization that firstly a monomer compound having an organic functional group (such as —OH or —COOH) is present and these organic functional groups are reacted with a compound containing the biomolecule-specific detection group only after the polymerization. This is basically known to the person skilled in the art.

With regard to preferred specific detection groups and biomolecules it may be referred to the above statements.

The removal of the colloidal particles of the photonic template crystals to obtain the porous photonic crystal structure may be effected through commonly known methods. For example, the colloidal particles may be removed by a solvent. In case of, for example, organic, polymeric particles, suitable organic solvents can be used. SiO₂ particles can be removed for example by hydrofluoric acid (HF).

According to a further aspect, the present invention relates to a device for the detection of biomolecules, comprising the above-described inventive responsive hydrogel having a porous photonic crystal structure.

With regard to the characteristics of the responsive hydrogel reference may be made to the above statements.

Also with regard to the biomolecules to be detected preferably with the device, reference may be made to the above embodiments.

The inventive apparatus for the detection of biomolecules can also have further elements which are customary for this type of devices, for example a signal converter and/or an electrical amplifier.

However, since according to the invention during the binding of the biomolecules to the detection groups the responsive hydrogel may show a significant shift of the peak position of the Bragg reflection in the wavelength range of visible light, it is also possible within the context of the present invention that the device has neither a signal converter nor an electrical amplifier.

According to a further aspect, the present invention relates to the use of the hydrogel as described above for the detection of biomolecules. The detection of biomolecules can be carried out isothermally.

The invention is described by the following examples in more detail.

### EXAMPLES

The preparation of the hydrogels in the form of porous photonic crystals was carried out via a template process. A photonic crystal comprising monodisperse particles serves as the template. For this purpose, monodispersed silica particles having a diameter of 400 nm were prepared according to the established “Stöber method” (as described in W. Stoeber et al., J. Colloid Interface Sci., 1968, 26, 62-69). These silica particles have been deposited vertically on specimen slides. The ethanolic silica dispersion was adjusted to a concentration of 2 wt.-percent by the addition of ethanol and water (medium: 80 wt.-% EtOH, 20 wt.-% ultra-pure water). Then the dispersion was filtered into a beaker (1 μm Acro Disk, Pall), the beaker was placed in a drying oven (40° C.) and a purified specimen slides made of soda-lime glass have been dipped into the dispersion. Within up to five days (depending on the amount of medium) the medium evaporated and the particles remained uniformly (like a crystal lattice) arranged on the surface. The resulting photonic template crystals had a vertical thickness of about 5 μm and showed pronounced opalescence. The coated specimen slide was then covered with another glass slide and the thus resulting shape has been sealed on three sides. Through the open side of a solution with the monomers, Irgacure 2010 as a UV initiator (1.5 wt.-% relative to the monomers) and water and ethanol as the solvent (total content of monomers and crosslinkers in the solution: 35 wt.-%) has been injected. The polymerization solution filled the interstices between the particles after the injection. Subsequently, the polymerization mold has been irradiated with UV light (emission maximum 365 nm, 400 W, Fa. Hoenle, type UVA Cube) and the hydrogel crosslinked.

The monomers used for the preparation of the hydrogels are listed in Table 1.

TABLE 1
Monomers used for the preparation of the hydrogels

| | HEMA | | OEGMA₃₀₀ | OEGMA₄₇₅ | MEO₂MA | OEGDMA₄₀₀ | OEGDMA₅₅₀ |
|---|---|---|---|---|---|---|---|
| Ex. 1 | 67 | mol % | 28 mol % | — | — | 5 mol % | — |
| Ex. 2 | 64 | mol % | 27 mol % | — | — | 9 mol % | — |
| Ex. 3 | 8 | mol % | — | 12 mol % | 65 mol % | — | 15 mol % |
| Ex. 4 | 7 | mol % | — | 11 mol % | 62 mol % | — | 20 mol % |

HEMA: hydroxyethyl methacrylate
OEGMA₃₀₀: oligo (ethylene glycol) methyl ether methacrylate, average number of ethoxy groups: 4.5
OEGMA₄₇₅: oligo (ethylene glycol) methyl ether methacrylate, average number of ethoxy groups: 7.5
MEO₂MA: di (ethylene glycol) methyl ether methacrylate
OEGDMA₄₀₀: oligo (ethylene glycol) dimethacrylate, serves as a crosslinker
OEGDMA₅₅₀: oligo (ethylene glycol) dimethacrylate, serves as a crosslinker

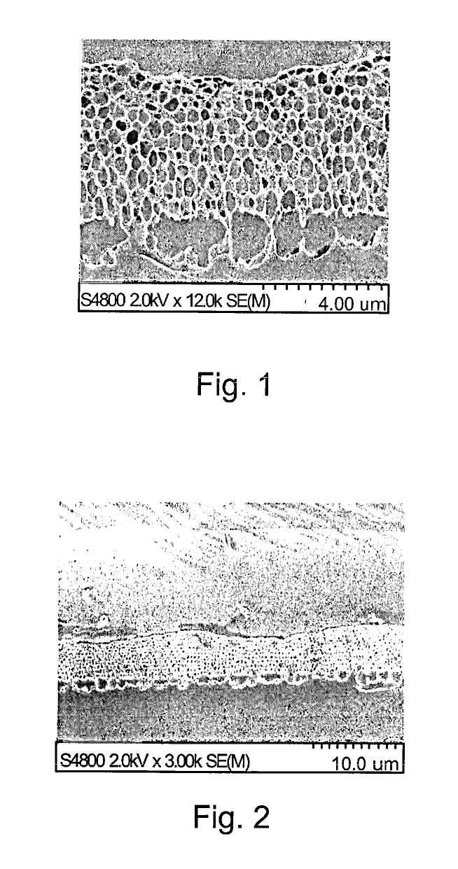

After removal of the mold a free standing hydrogel was obtained, which was then placed for about 30 min in 2% HF solution to dissolve the SiO₂ particles. Since the template particles were so tightly packed that they touch each other, the contact areas remained free of monomer. Thus at the intersections channels arose between the individual cavities. The cavities have a diameter of several hundred nanometers and were arranged so that their structure resembles the structure of crystal planes. Thus the responsive hydrogel is present in the form of a porous photonic crystal. The structures were detected by scanning electron microscopy. This is shown in FIGS. 1 and 2.

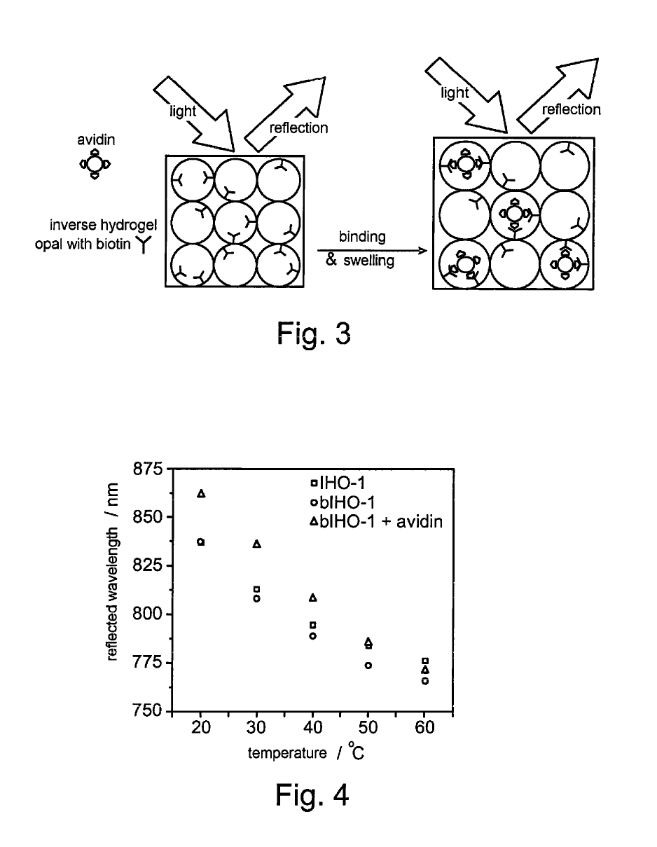

As a model system for the detection of biological detection, the binding pair biotin/avidin was used. Biotin served as a detection group, which is immobilized in the porous photonic crystal, avidin is the analyte to be detected. Up to a molecular weight of about 66 kDa, it is a biopolymer. Up to four biotin units bind selectively and with a high binding constant (K-1015) to an avidin molecule. For the detection of avidin biotin was covalently coupled to the porous photonic crystal. This was achieved via a polymer-analogous Steglich-esterfication of the carboxyl group of biotin with the hydroxyl group of the hydroxyethyl methacrylate. An excess of biotin (1:1 w/w regarding the dry hydrogel) was dissolved in warm, dry dimethylformamide (DMF) and subsequently cooled. The hydrogel film was conditioned in dry DMF and added to the biotin solution. Dicyclohexylcarbodiimide (1.2 eq to biotin) was dissolved in dry dichloromethane (DCM) and 0.1 eq of dimethylaminopyridine (DMAP) was added. Both solutions were combined and allowed to react overnight. Subsequently, the porous photonic crystal was washed with DCM, DMF and ultrapure water. The immobilization can also be carried out with other coupling agents known in the literature such as 1-ethyl-3-(3-dimethylaminopropyl) carbodiimide (EDC), 1-hydroxybenzotriazole (HOBt) or intermediates activated with N-hydroxysuccinimide (NHS). In such a case the reaction conditions should be adjusted accordingly. The amount of accessible, coupled biotin was determined in the hydrogel using the established HABA/avidin-assays to approximately 0.1% of the hydroxyl groups, which can be coupled. Upon addition of hydrophilic avidin to the biotinylated porous photonic crystal the detection group and the analyte bound to each other, whereby the hydrogel swelled. Thus, the distances of the honeycomb layers and thus the reflected color are altered. This process is illustrated schematically in FIG. 3. The left half of FIG. 3 shows a hydrogel with biotin as biomolecule-specific detection groups, wherein the hydrogel exhibits a Bragg reflection due to its porous photonic crystal structure. If avidin is supplied, it binds to the detection groups. The hydrogel swells, however, the photonic crystal structure will remain, so that further a Bragg reflection is observed. This is illustrated in the right half of FIG. 3. By swelling the hydrogel, however, the position of the Bragg peak shifts. The swelling of the hydrogel was thereby enhanced by the thermoresponsive polymer, since the swelling is more pronounced than in a non-responsive hydrogel. The change in the hydrophilicity by the bonding process altered the phase behavior of the polymer and provided for an increased water uptake or release, similar to the reaction of these polymers to increase or decrease the temperature, with the difference that this was done at a constant temperature. Thus, the porous photonic hydrogel crystal responded with a pronounced color change than it would be the case with a purely hydrophilic system.

In FIG. 4, the wavelengths of color reflection of a responsive porous photonic crystal are plotted. In FIG. 3 “IHO-1” refers to the not yet biotinylated porous photonic crystal, “biHO-1” to the biotinylated porous photonic crystal before avidin-addition and “biHO-1+avidin” to the biotinylated porous photonic crystal after the addition of avidin. The porous photonic hydrogel crystal was prepared according to Ex. 1 under the conditions mentioned above. The accessible biotin content of the film after Steglich esterfication with DCC as coupling agent was 0.01% relative to the existing hydroxyl groups. It has been found that after addition of avidin to biotinylated inverse opal (triangles), the peak wavelengths are red-shifted. The effect is most pronounced at room temperature, which is advantageous for a method of the detection of biomolecules.

1. A responsive hydrogel, which is chemically crosslinked, has a porous photonic crystal structure, and contains biomolecule-specific detection groups.

2. The responsive hydrogel according to claim 1, namely a thermoresponsive hydrogel.

3. The responsive hydrogel according to claim 1, wherein the responsive hydrogel swells or shrinks when biomolecules bind to the specific detection groups.

4. The responsive hydrogel according to claim 1, wherein the responsive hydrogel exhibits a volume phase transition when the biomolecules bind to the specific detection groups.

5. The responsive hydrogel according to claim 1, wherein the porous photonic crystal structure has interconnected cavities.

6. The responsive hydrogel according to claim 1, comprising one or more of the following monomer units: 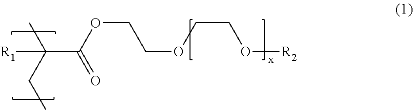
wherein;
R₁═H, alkyl,
R₂═H, alkyl, —(CH₂)ₙ—COOH with n=1 to 12 or a salt thereof,
x=0-50, 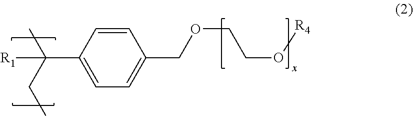
wherein;
R₁═H, alkyl;
R₄═H, alkyl, —(CH₂)ₙ—COOH with n=1 to 12 or a salt thereof;
x=0-50, 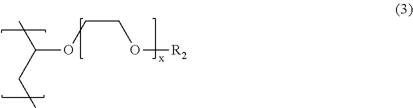
wherein;
R₂═H, alkyl, —(CH₂)ₙ—COOH with n=1 to 12 or a salt thereof,
x=0-50, 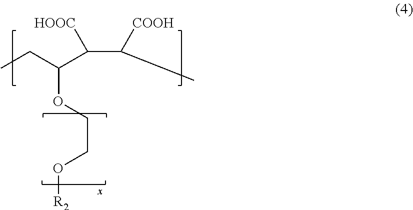
wherein;
R₂═H, alkyl, —(CH₂)ₙ—COOH with n=1 to 12 or a salt thereof;
x=0-50, 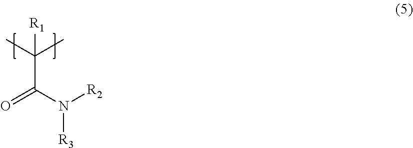
wherein;
R₁═H, alkyl;
R₂ and R₃ independently of each other=H, alkyl, allyl, 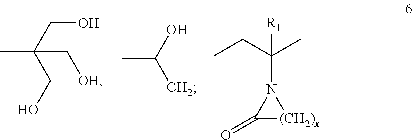
wherein;
R₁═H, alkyl;
x=1-6, 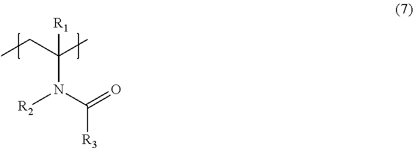
wherein;
R₁, R₂, R₃ independently of each other=H, alkyl; 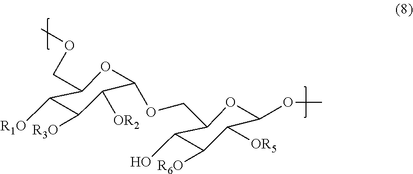
R₁═H or a branch of the polysaccharide chain
R₂, R₃, R₅, R₆ independently of each other=H, alkyl, allyl, —(CH₂)ₙ—COOH with
n=1-12 or a salt thereof,
(9)
polysaccharide units, 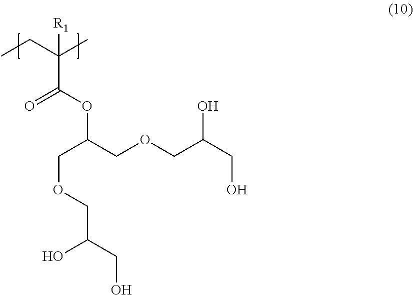
wherein;
R₁═H, alkyl, 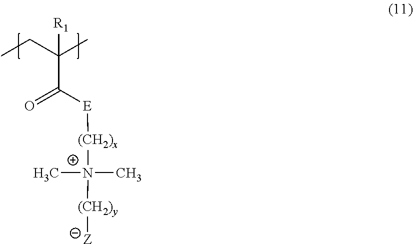
wherein;
R₁═H, alkyl,
E=O or NH,
x, y independently of each other may acquire values of from 1 to 12 Z═SO₃, COO, or PO₃.

7. The responsive hydrogel according to claim 1, comprising the following monomer units (1a) and (1b): 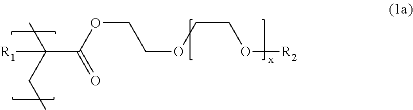
wherein;
R₁═H, alkyl;
R₂═H, alkyl, —(CH₂)ₙ—COOH with n=1-12 or a salt thereof;
x=0 or 1 or 2, and 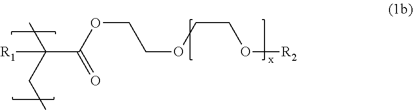
wherein;
R₁═H, alkyl;
R₂═H, alkyl, —(CH₂)ₙ—COOH with n=1-12 or a salt thereof;
x=3-50.

8. The responsive hydrogel according to claim 7, wherein the monomer units (1a) are present in an amount of 30-90 mol % based on the total amount of the monomers, and the monomer units (1b) are present in an amount of 2-40 mol %, based on the total amount of monomers.

9. The responsive hydrogel according to claim 1, wherein the chemical crosslinking is performed by preparing the hydrogel in the presence of crosslinkable monomers, and the crosslinkable monomers are present in an amount of 2-20 mol %, based on the total amount of monomers.

10. The responsive hydrogel according to claim 1, wherein the biomolecule-specific detection groups are selected from antibodies, F_{ab} fragments of antibodies, enzymes, enzyme fragments, coenzymes, peptides, prosthetic groups, aptamers, DNA single strands and RNA single strands.

11. The responsive hydrogel according to claim 1, wherein the biomolecules are selected from biooligomeres, biopolymers and biological particles.

12. A process for preparing a chemically crosslinked, responsive hydrogel having a porous photonic crystal structure, comprising:
(i) providing a photonic template crystal of colloidal particles,
(ii) introduction of monomers into the interstices being present between the colloidal particles,
(iii) polymerization of the monomers, thereby obtaining a chemically crosslinked, responsive hydrogel,
(iv) removal of the colloidal particles of photonic template crystal, thereby obtaining the porous photonic crystal structure.

13. The method of claim 12, wherein the colloidal particles have an average diameter in the range of 600 nm to 100 nm.

14. The method of claim 12, wherein one of the monomers in step (ii) comprises a biomolecule-specific detection group, or a biomolecule-specific detection group is covalently bound to the hydrogel after polymerization.

15. The method according to claim 12, wherein the responsive hydrogel is the responsive hydrogel.

16. An apparatus for detecting biomolecules, comprising the responsive hydrogel according to claim 1.

17. (canceled)
